(19) United States
(12) Patent Application Publication
(10) Pub. No.: US 2005/0288516 A1
(43) Pub. Date: Dec. 29, 2005

(54) Use of a device or devices, such as a convergent divergent funnel mixer, to optimize the available reaction volume, the raw material feed ratios and the weight hourly space velocity in a tube reactor

(76) Inventor: Jack S. Warren, Cumming, GA (US)

Correspondence Address: Jack S. Warren, 2825 Stonehenge Court, Cumming, GA, 30041, US

(21) Appl. No.: 10/878,077

(22) Filed: Jun. 28, 2004

(51) Int. Cl. C07D 261/18

(52) U.S. Cl. 548/248

(57) ABSTRACT

A method of using an apparatus, device or devices, such as a convergent divergent funnel, as a mixer for the feed material, to optimize the available reaction volume (ARV), the raw material feed ratios (R1:R2) and the weight hourly space velocity (WHSV), to produce organic compounds, in a tube reactor. These organic compounds include, but are not limited to: acids, aldehydes, amides, esters, ethers and ketones, which are useful as chemical, agricultural and pharmaceutical intermediates, pharmaceuticals, agricultural agents, herbicides, insecticides, pesticides, insect repellents, animal repellents, plasticizers, dye carriers and as flavor and/or fragrance ingredients.

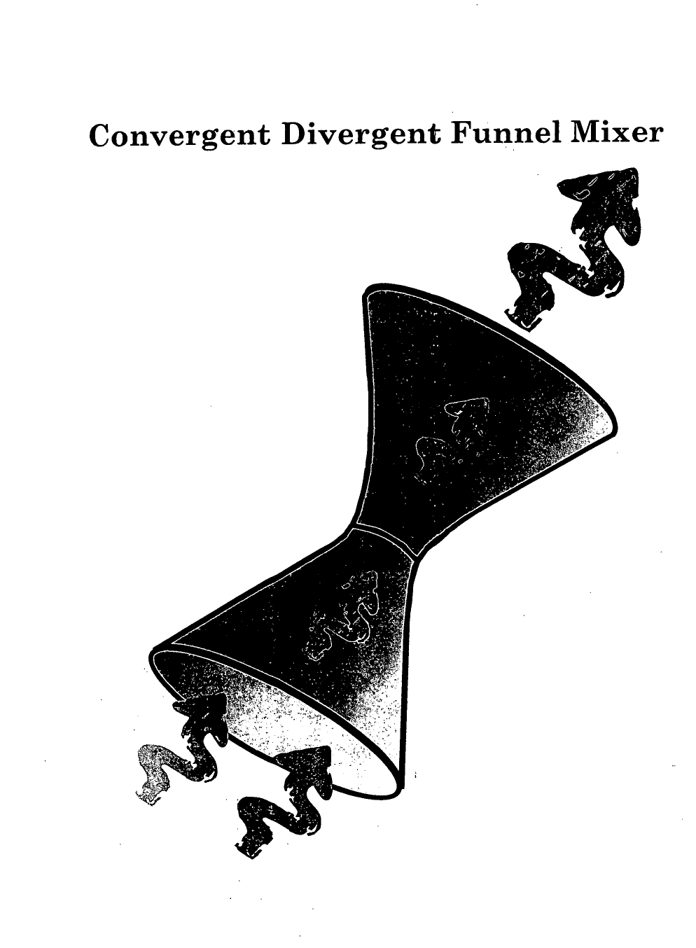

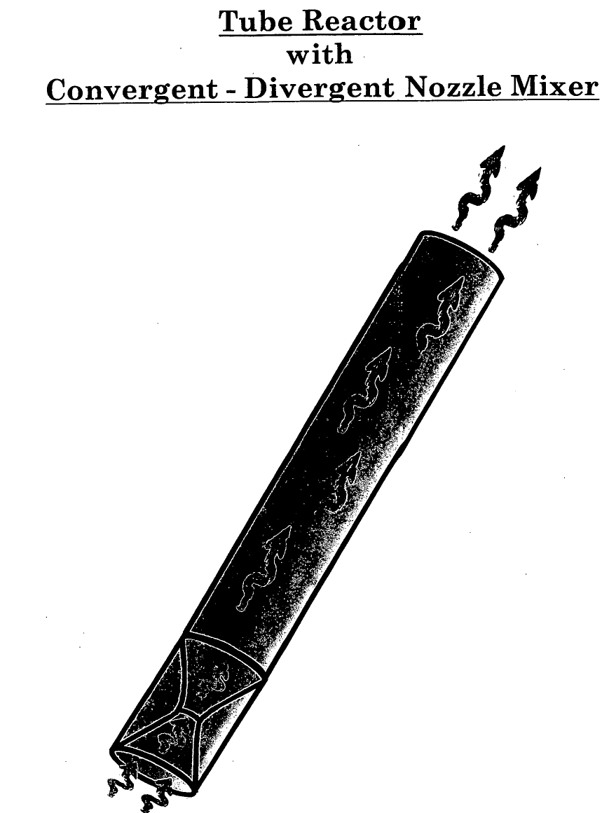

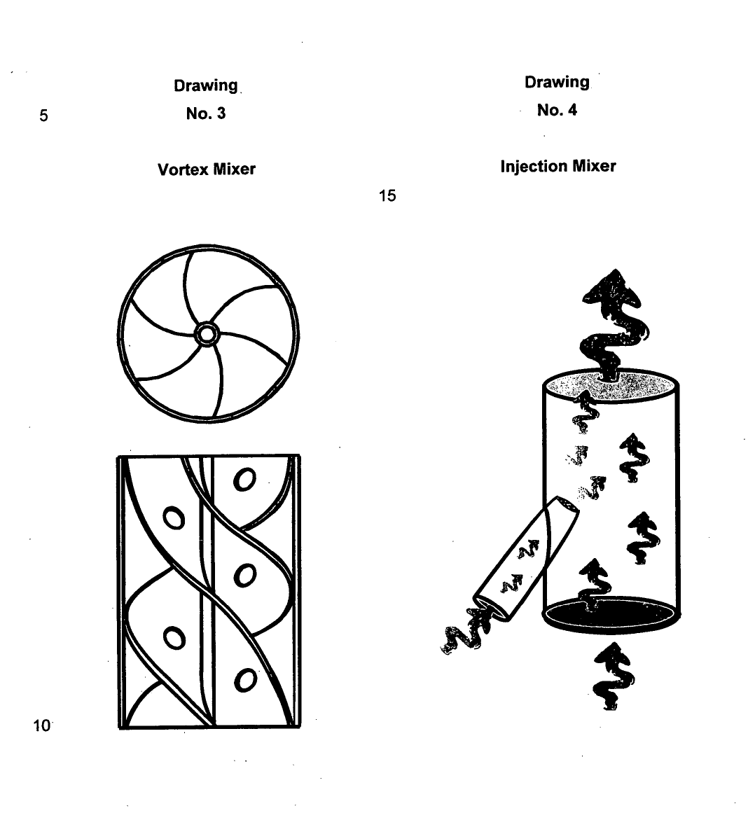

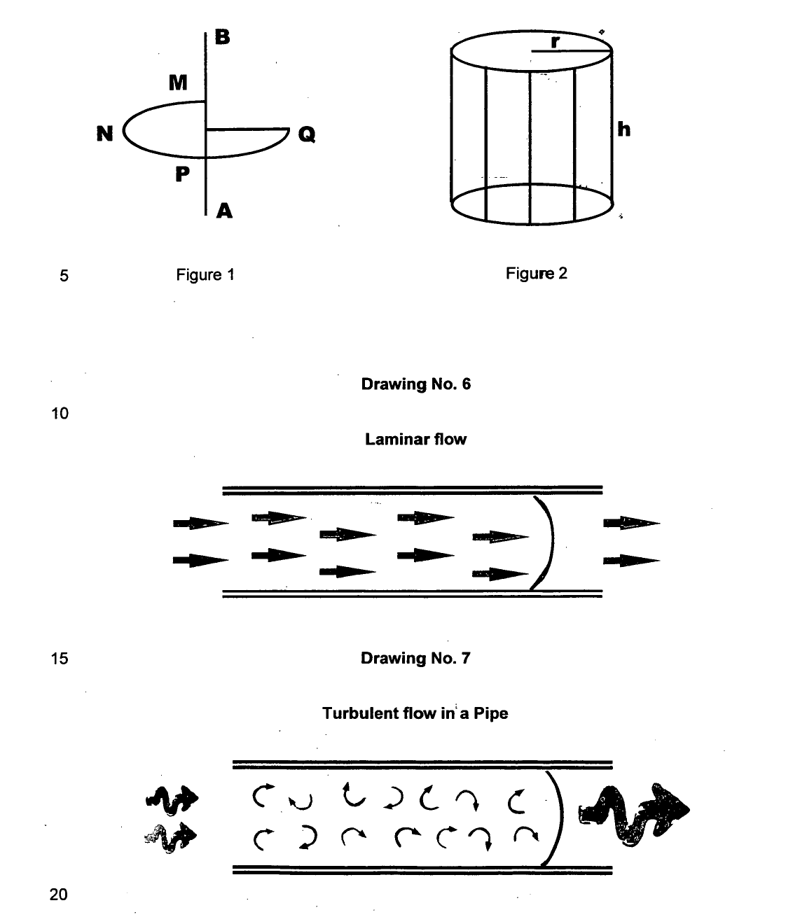

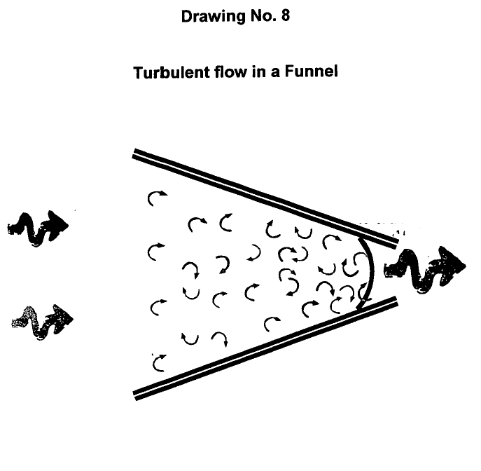

### BACKGROUND OF THE INVENTION

1. Field of the Invention

The present invention relates generally to an enhanced method and improved apparatus, device or devices, for the preparation of various organic compounds, such as: acids, aldehydes, amides, esters, ethers, and ketones. The invention relates more particularly to the use of a method and an apparatus, device or devices, such as a convergent divergent funnel mixer/reactor, for the production of aldehydes, amides, esters and ketones and, most particularly to the use of a convergent divergent funnel mixer/reactor for preparing aldehydes, such as meta-tolualdehyde (MTA), amides, such as N,N′-di-(ethyl)-meta-toluamide (DEET), esters, such as benzyl benzoate and ketones, such as methyl nonyl ketone (MNK), methyl cyclopropyl ketone (MCPK) and di-isopropyl ketone (DIPK). The invention also relates to using such organic compounds in the preparation of chemical, agricultural and pharmaceutical intermediates, pharmaceuticals, agricultural agents, herbicides, insecticides, pesticides, insect repellents, animal repellents, plasticizers, dye carriers and as flavor and/or fragrance ingredients.

2. Description of the Prior Art

Numerous literature references cite and disclose various well-known processes for the preparation of ketones. These processes include oxidation of secondary alcohols; Friedel-Crafts acylation; reaction of acid chlorides with organic cadmium compounds; acetoacetic ester synthesis and decarboxylation from acids, among others.

Text and literature references also detail problems associated with these processes to produce ketones. These include problems such as the unavailability and/or high cost of raw materials, the requirement of multi-stage processing, the low conversion of raw materials and/or the low selectivity of the desired ketones, and the production of corrosive or hard-to-separate products.

Most ketone manufacturing processes include the reaction of various reactants at specified temperature and pressure ranges in the presence of a catalyst. For example,

U.S. Pat. No. 4,528,400 discloses a method of preparing unsymmetrical ketones by a catalytic vapor phase reaction using reactants such as ketones with carboxylic acids in the presence of a ceria-alumina catalyst. U.S. Pat. No. 4,874,899 involves the preparation of unsaturated and saturated ketones in the presence of a catalyst such as a zeolite, a phosphate having a zeolite structure and/or a B, Ce, Fe, Zr or Sr phosphate. U.S. Pat. No. 4,570,021 relates to the preparation of ketones utilizing a ceria-alumina catalyst. U.S. Pat. No. 4,060,555 discloses the production of a class of aliphatic ketones in the presence of Deacon Catalysts. U.S. Pat. No. 3,966,822 discloses the preparation of ketones from aldehydes in the presence of zirconium oxide and various other catalysts. U.S. Pat. No. 3,466,334 discloses synthesis of ketones from an aldehyde and an acid in the presence of a catalyst comprised of an alumina-supported oxidized form of lithium. U.S. Pat. No. 3,453,331 discloses a process for the synthesis of ketones from aldehydes using various alumina-supported oxidized forms of various metals. German Patent Application, No. P 36 37 788.0 discloses a process for the preparation of a ketone in the presence of catalysts such as ZnO and/or CeO₂ doped on aluminum oxide (Al₂ O₃).

U.S. Pat. No. 6,369,276 B1; U.S. Pat. No. 6,392,099 B1; U.S. Pat. No. 6,482,991 B2; U.S. Pat. No. 6,495,696 and U.S. Pat. No. 6,545,185 address the need in the art for a catalyst or catalyst structure useful in the production of ketones and aldehydes which not only allows the reaction to proceed, but which also optimizes the conversion and selectivity of the reaction to the desired ketone or aldehyde and permits conversion and selectivity for various catalyst structures to be reasonably predicted. They also address the method of making such a catalyst and for using such a catalyst in the production of ketones and aldehydes. The catalyst structure includes a substantial theoretical monolayer (TML) of catalyst on a catalyst support to optimize yield and weight hourly space velocities (WHSV). As used with these patents, the term theoretical monolayer (TML) is a thin film or layer of a material (catalyst) applied to a surface (catalyst support) at a thickness of one molecule and a substantial theoretical monolayer means plus or minus 10% of a theoretical monolayer.

These patents also describe the use of preferably conventional stainless steel tube reactors, where the available reaction volume, is filled with various combinations of an inert filler material, and a theoretical monolayer catalyst. Available Reaction Volume (ARV) is the total (inside) volume of the tube reactor. The inert filler material is comprised of glass beads, stainless steel beads, lava rock and sand, among possible others. The distribution of the catalyst within the available reaction volume can vary. Preferably, however, the method and use of these patents claim, the bottom ⅓ of the reactor is filled with inert material in the form of glass beads, the middle third of the reactor is filled with a catalyst and the top ⅓ of the reactor could be empty or filled with glass beads or another inert material. U.S. Pat. No. 4,570,021 and U.S. Pat. No. 4,528,400 also describe the use of glass beads, in a tube reactor, ahead of and behind the catalyst zone.

International Publication Number WO 02/36559 A2 discloses, in the preferred embodiment, the invention of a process for the production of N,N-di(ethyl)-meta-toluamide comprising: (a) reacting meta-xylene and oxygen to form meta-toluic acid, wherein the reaction occurs in the liquid or vapor phase and in the presence of a first catalyst; (b) separating the meta-toluic acid from the mixture formed in step (a), wherein the meta-toluic acid is maintained in a liquid or vapor phase; and (c) reacting the meta-toluic acid with diethylamine to form N,N-di(ethyl)-meta-toluamide, wherein the reaction occurs in the vapor phase and in the presence of a second catalyst, in one or more tube reactors, using a theoretical monolayer catalyst and inert filler material.

Numerous patents have been issued for converging and/or diverging nozzles, with a wide variety of applications, such as laser devices, venting means for nuclear reactors, combustion and/or turbo-jet mufflers, flow bodies, animal feed device, reactors for the production of salts and fast quenching reactors, among others. U.S. Pat. No. 6,284,189 B1 describes a nozzle device to inject oxygen and technological gases used in metallurgical processing of metal melting, the nozzle being suitable to emit a gassy flow at supersonic velocity, the nozzle having a conformation symmetrical to a central axis (x) defined by a throat arranged between the inlet and the outlet, the throat defining an upstream part with a convergent development and a downstream part with a divergent development which ends in the outlet mouth, the nozzle with the convergent/divergent development having a geometry such that the fall in pressure of the gassy flow from inlet to outlet has a hyperbolic tangent development. It also describes a dimensioning method for the nozzle as above, the method providing an inverse dimensioning approach wherein the geometry of the nozzle is adapted to the natural profile of the fall in pressure of the gassy flow according to a hyperbolic tangent development, thus obtaining an optimum variation of the aerodynamic parameters according to the natural laws of expansion.

However, the dimensioning method for the converging diverging nozzle is meant to optimize the gassy flow at supersonic velocity and does not address the need in the art for subsonic irregular or turbulent flow in the converging diverging transition section to promote mixing of raw materials, which are then used in a chemical process.

U.S. Pat. No. 6,437,001 B1 describes the use of an unsymmetrical ketone as an active ingredient to repel insects; however, it does not address the need for a more cost effective manufacturing process for these active ingredients, to compete with existing repellent products.

U.S. Pat. No. 6,524,605 B1 describes the use of a Monoterpenoids, such as Nepetalactone, derived from a biorational source, such as a plant volatile; but does not address the need for a more cost effective chemical manufacturing process for these active ingredients; to repel arthropods, such as termites.

In examples, U.S. Pat. No. 6,369,276 B1 and many others; describe the ratio of raw materials, which makes up the feed stream or feed material, as preferably in the range of 2:1 to 20:1; more preferably, the ratio of about 3:1 to 8:1 and most preferably within a range of 3:1 to 5:1. The most preferred ratio is about 4:1. Using an excess of the least expensive raw material is common practice in the chemical industry; with a driving force being; to “use-up” or consume ˜100% of the most expensive raw material. However, this practice results in excess production of co-products and/or the separation and recovery of the un-reacted raw material that passes through the reactor. This excess raw material ratio also has an effect on the optimum WHSV.

Although a great deal of attention has been given to the use of convergent and/or divergent funnels and nozzles; to catalyst and catalyst structure, to the method of making a catalyst; to the ratio of the raw material feed; to process parameters, such as temperature and pressure; in connection with the production of acids, aldehydes, amides, esters, ethers and ketones; little, if any, attention has been given to the inert filler material or to the distribution of the catalyst and inert filler material inside the available reaction volume (ARV), of a tube reactor, to optimize yield (raw material conversion and selectivity) to the desired product. In addition, little attention has been given to raw material mixing, as well as to the optimum (theoretical stoichiometric) raw material ratio (r₁ₜ:r₂ₜ) on the weight hourly space velocity in a tube reactor process.

Accordingly, there is a need in the art for an enhanced method and apparatus, device or devices, to provide mixing of the feed materials, to optimize the available reaction volume (ARV); the raw material feed ratios (R1:R2) and the weight hourly space velocity (WHSV) which provides for a significantly improved production rate and cost of organic compounds including: acids, aldehydes, amides, esters, ethers and ketones; and particularly, esters, such as benzyl benzoate, amides, such as N,N-di(ethyl)-meta-toluamide (DEET) and ketones, such as methyl nonyl ketone (MNK), methyl cyclopropyl ketone (MCPK) and di-isopropyl ketone (DIPK); which are useful as chemical, agricultural and pharmaceutical intermediates, pharmaceuticals, agricultural agents, herbicides, insecticides, pesticides, insect repellents, animal repellents, plasticizers, dye carriers and as flavor and/or fragrance ingredients.

### SUMMARY OF THE INVENTION

In contrast to the prior art, the present invention relates generally to a method and apparatus, device or devices, for the preparation of various organic compounds, such as: acids, aldehydes, amides, esters, ethers, and ketones. The invention relates more particularly to the use of a device or devices, such as a convergent divergent funnel mixer/reactor, for the production of aldehydes, amides, esters and ketones and most particularly to the use of a device or devices, such as a convergent divergent funnel mixer/reactor, for preparing aldehydes, such as meta-tolualdehyde (MTA), amides, such as N,N-di(ethyl)-meta-toluamide (DEET); esters, such as benzyl benzoate and ketones, such as methyl nonyl ketone (MNK), methyl cyclopropyl ketone (MCPK) and di-isopropyl ketone (DIPK); which overcomes limitations of the prior art. The invention also relates to using such aldehyde, amide, ester and ketone preparation in the preparation of insect repellents, animal repellents, chemical intermediates, herbicidal or other agricultural compounds and as flavor and/or fragrances ingredients.

Specifically, the method and apparatus, device or devices, of the present invention utilizes readily available and inexpensive raw materials, results in high conversion and selectivity and provides for increased production of the desired products. Generally, the raw materials used in the method and apparatus of the present invention include: aromatic or aliphatic hydrocarbons, acids or aldehydes or their derivatives, alcohols, amines, carboxylic acids, oxygen or an oxygen source. More specifically, the present invention involves the preparation of aldehydes, such as meta-tolualdehyde (MTA), amides, such as N,N-di(ethyl)-meta-toluamide (DEET); esters, such as benzyl benzoate and ketones, such as methyl nonyl ketone (MNK), methyl cyclopropyl ketone (MCPK) and di-isopropyl ketone (DIPK), utilizing a tube reactor provided with a suitable catalyst. For purpose of this application and method, the catalyst is a super-layer catalyst; defined as greater than 110% of a theoretical mono-layer. The preferred raw materials or feed materials include, but or not limited to: benzoic acid, benzyl alcohol, meta-toluic acid (MTA), diethylamine (DEA), decanoic acid, cyclopropylaldehyde or its derivatives (such as cyclopropanecarboxylic acid), butyric acid and acetic acid which are readily available through processes known in the art. Depending on the desired organic compound, the properly selected, gas phase raw materials are fed into and through a device or devices, such as a convergent divergent funnel mixer, attached to a tube reactor, where they are exposed to a catalyst and react to produce the desire product.

By adding a device or devices, such as a convergent divergent funnel, as a raw material mixer, to a tube reactor process, the present invention allows for a 100-200% increase in the available reaction volume (ARV). Previous art requires the use of an inert filler material, inside the reactor, ahead of and an inert filler material or empty space behind the catalyst. This inert filler material, which occupies reaction volume, is comprised of glass beads, stainless steel beads, lava rock and sand, among possible others. The distribution of the inert filler material within the available reaction volume (ARV) can vary greatly; however, previous patents “preferably” require the bottom ⅓ and the top ⅓ of the available reaction volume (ARV) to be empty or filled an inert material. The purpose of this inert filler material, before the catalyst, is to provide a zone for mixing and/or heating of the raw materials before they reach the catalyst.

For some tube reactor processes, it could be impossible to increase the WHSV, because the inert filler material zone does not provide sufficient volume and time to allow for complete mixing and heating of the raw materials before they reach the catalyst. For example; Using a pre-mixed liquid feed at 30° C., WHSV=20; in a nine (9′) foot long, six (6″) inch diameter, tube reactor, with a configuration of ⅓ glass beads, ⅓ catalyst (48.5 lbs/ft³) and ⅓ glass beads, reaction temperature of 330° C.; would need ten (10) pounds of raw material feed to be heated (ΔH=˜300° C.) and mixed, in the 36 inch long inert filler zone before the catalyst, in only twenty (20) seconds! Feeding separate pre-heated, raw materials would help; however the mixing could still be incomplete.

Pre-heating a theoretical stoichiometric ratio (TSR) of raw materials and feeding these raw materials into a device, such as the converging section of a converging diverging funnel mixer, would allow for complete mixing and a greatly increased WHSV.

During catalyst change-over or routine reactor maintenance, when the reactor is reloaded with new inert filler material and catalyst, the flow of material through the available reaction volume is, more often than not, changed. The new inert filler material can be more or less tightly packed or have different surface characteristics, which would causes the flow channels (voids) within the inert filler material zone and catalyst zone to change. Changing conditions within the inert filler material zone are a disadvantage to the process and have a negative effect on process controls, such as raw material mixing, temperature and pressure, and on the actual conversion and yield.

By the addition of a mixing device or devices, such as a converging diverging funnel mixer, to the tube reactor and removal of the inert filler material before the catalyst, the volume of the catalyst zone can be increased by as much as 100%. This allows for approximately 60-70% of the total available reaction volume (ARV) to be loaded with catalyst. The use of a device or devices, such as a converging diverging funnel mixer, also allows for constant, measurable and controllable mixing parameters.

After the catalyst zone, the inert filler material is used to hold the catalyst in position, provides a head-space, or a reaction quenching and cooling zone for the products and co-products. Depending on the reaction conditions, determining the optimum requirements for the inert filler material volume, after the catalyst zone, could allow for the loading or addition of an additional 30-40% of catalyst, in the available reaction volume.

In the preferred embodiment and method of the present invention, the reactor is a gas/vapor phase tube reactor and attached to the divergent section of a convergent divergent funnel mixer, in contrast to a condensation reactor or a batch stirred (mixed) reactor. Since some chemical reactions will be exothermic and others will be endothermic, the tube reactor and the convergent divergent funnel mixer are provided with an external heat and cooling source, as well as insulation. The reactant materials are pre-heated to the gas/vapor phase. Pre-heating equipment is available from companies skilled in the art, such AccuTherm, Inc. Monroe City, Mo., U.S.A.

Further, in the enhanced method and improved apparatus of the present invention the reactant materials, in a theoretical stoichiometric ratio (TSR), are fed into a device or devices, such as the convergent section of the funnel mixer with sufficient pressure to cause flow through the convergent divergent transition section of the mixer, where the significantly increased velocity and turbulent flow created by the converging section of the funnel causes mixing of the raw material. The theoretical stoichiometric raw material mixture then pass through the divergent section of the funnel mixer, into the reactor, which contains sufficient catalyst to fill the available reaction volume (ARV). With this configuration, it is possible to dramatically reduce excess raw material consumption, minimize production of co-products, maximize use of the reactor capacity and greatly increase the WHSV. This results in significantly increased production rates and lower cost.

An object of the present invention is to provide for using the above described enhanced method and improved apparatus, device or devices, for the preparation of: chemical, agricultural and pharmaceutical intermediates, pharmaceuticals, agricultural agents, herbicides, insecticides, pesticides, insect repellents, animal repellents, plasticizers, dye carriers and flavor and/or fragrance ingredients.

Accordingly, an object of the present invention is to provide an enhanced method and improved apparatus, device or devices, for the preparation of aldehydes, amides, esters and ketones and in particular aldehydes, such as meta-tolualdehyde (MTA), amides, such as N,N-di(ethyl)-meta-toluamide (DEET), esters, such as benzyl benzoate and ketones, such as methyl nonyl ketone (MNK), methyl cyclopropyl ketone (MCPK) and di-isopropyl ketone (DIPK).

A further object of the present invention is to provide an enhanced method and improved apparatus, device or devices, for the preparation of MTA, DEET, Benzyl Benzoate, MNK, MCPK or DIPK at high conversion rates and high selectivity, with a minimum of undesirable co-products.

A still further object of the present invention is to provide an enhanced method and improved apparatus, device or devices, for the preparation of meta-tolualdehyde (MTA), N,N-di(ethyl)-meta-toluamide (DEET), benzyl benzoate, methyl nonyl ketone (MNK), methyl cyclopropyl ketone (MCPK) and di-isopropyl ketone (DIPK), at dramatically increased production rates and lower cost.

These and other objects of the present invention will become apparent with reference to the drawings, the definitions, the description of the preferred embodiment and the appended claims.

### DESCRIPTION OF THE PREFERRED EMBODIMENT

Reference is made to Drawing 1, where the apparatus, device or devices, is a symmetrical converging diverging funnel mixer with an inside diameter of six (6) inch, at the mouth; and a transition diameter of three-fourth (¾) inch, between the converging and diverging sections. The diagram shows flow of two (2) separate raw materials, which have been pre-heated to gas phase. The two materials enter into the converging section in a substantial theoretical stoichiometric ratio, at a velocity (v₁). The flow velocity accelerates, as the raw materials approach and passes into the transition section at a velocity (v₂), according to the formula:
v₂=v₁*(r₁/r₂)²

This exponential increase in velocity causes turbulent flow, and results in a mixing of the two raw materials. Pressure in the funnel mixer and reactor is controlled to maintain the raw material feed in a gas phase.

Reference is made to Drawing 2, which shows a stainless steel gas phase tube reactor connected to a mixing device. The reactor is connected to the diverging section of a symmetrical converging diverging funnel mixer, which is constructed of hastelloy alloy.

As the mixed raw material feed passes out of the diverging section, it enters into the catalyst zone in the tube reactors available reaction volume (ARV). For this example, the total available reaction volume (ARV) of the tube reactor is filled with a super-layer catalyst, suitable for the process. External heating or cooling and insulation are used to maintain the catalyst zone at the appropriate reaction temperature. In the presence of heat and catalyst, the theoretical stoichiometric mixed ratio raw materials react to form the desired product.

The desired product and co-products then pass out of the tube reactor into product receiver equipment for recovery, separation and distillation.

### EXAMPLE

Reference is made to Drawing 1, where the apparatus, device or devices, is a symmetrical converging diverging funnel mixer with an inside radius of three (3) inch, at the mouth; and a transition radius three-eights (⅜) inch, between the converging and diverging sections. The diagram shows flow of two (2) separate gas phase raw materials (acetic acid and decanoic acid), which have been pre-heated to ˜300° C. The two acids enter into the converging section in a substantial theoretical stoichiometric ratio of 1.4:1.0, at a flow velocity (v₁) of 20 pounds per minute. The flow velocity accelerates, as acids approach; pass into and through the transition section at a velocity (v₂) of ˜1280 pounds per minute, according to the formula:
v₂=v₁*(r₁/r₂)²

This exponential increase in velocity causes turbulent flow, and results in a complete stoichiometric mixings of the two acids. Pressure in the funnel mixer and reactor is controlled at 120-150 psig, to maintain the mixed acids in a gas phase.

Reference is made to Drawing 2, which shows a six (6) inch diameter, ten (10) foot long stainless steel gas phase tube reactor connected to the diverging section of a symmetrical converging diverging funnel mixer, which is constructed of hastelloy alloy.

As the mixed acids pass through and out of the transition section of the converging diverging mixer; the flow velocity (V₃) decelerates; to ˜20 pounds per minute, according to the formula:
v₃=v₂*(r₂/r₃)²

The mixed acids enter into the catalyst zone in the tube reactors available reaction volume (ARV) (WHSV=14), which is filled with ˜82 pounds of a CeO₂/Al₂O₃ super-layer catalyst with a bulk density of 42.5 lb/ft3. External heating or cooling and insulation are used to maintain the catalyst zone at ˜305° C. In the presence of heat and catalyst, the theoretical stoichiometric mixed acids react to form a crude mixture: methyl nonyl ketone (MNK) and the corresponding co-products.

The crude MNK and co-product mixture then pass out of the tube reactor into a product receiver for recovery, separation and distillation. Conversion of the raw material feed acids is typically 97%±, with selectivity to MNK, the unsymmetrical ketone, of 90%±.

### EXAMPLE

Reference is made to Drawing 1, where the apparatus, device or devices, is a symmetrical converging diverging funnel mixer with an inside radius of three (3) inch, at the mouth; and a transition radius three-eights (⅜) inch, between the converging and diverging sections. The diagram shows flow of two (2) separate gas phase raw materials (acetic acid and cyclopropanecarboxylic acid), which have been pre-heated to ˜310° C. The two acids enter into the converging section in a substantial theoretical stoichiometric ratio of 1.6:1.0, at a flow velocity (v₁) of 30 lb/min [Re=1500]. The flow velocity accelerates as the acids approach; pass into and through the transition section, at a velocity (v₂) of ˜1900 lb/min [Re=3500], according to the formula:
v₂=v₁*(r₁/r₂)²

This exponential increase in velocity causes turbulent flow [Re=3500], and results in a complete stoichiometric mixings of the two acids. Pressure in the funnel mixer and reactor is controlled at 120-150 psig, to maintain the mixed acids in a gas phase.

Reference is made to Drawing 2, which shows a six (6) inch diameter, ten (10) foot long stainless steel gas phase tube reactor connected to the diverging section of a symmetrical converging diverging funnel mixer, which is constructed of hastelloy alloy.

As the mixed acids pass through and out of the transition section of the converging diverging mixer; the flow velocity (v₃) decelerates; to ˜30 lb/min [Re=1800], according to the formula:
v₃=v₂*(r₂/r₃)²

The mixed acids enter into the catalyst zone in the tube reactors available reaction volume (ARV) (WHSV=20), which is filled with ˜90 pounds of a CeO₂/Al₂O₃ super-layer-catalyst with a bulk density of 46.8 lb/ft3. External heating or cooling and insulation are used to maintain the catalyst zone at ˜310° C. In the presence of heat and catalyst, the theoretical stoichiometric mixed acids react to form a crude mixture: methyl cyclopropyl ketone (MCPK) and the corresponding co-products.

The crude MCPK and co-product mixture then pass out of the tube reactor into a product receiver for recovery, separation and distillation. Conversion of the raw material feed acids is typically 98%+, with selectivity to MCPK, the unsymmetrical ketone, of 89%+.

### EXAMPLE

The ketones produced by the improved apparatus and enhanced method of the present invention can be distilled and combined with other processes to produce various herbicidal or other agricultural compounds. Preferably, the ketone production method of the present invention can be used, in combination with other process steps, to prepare such a compound of the formula (I) 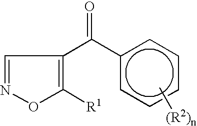
wherein:

R¹ is cycloalkyl having from three to six ring carbon atoms which is un-substituted or which has one or more substituents selected from the group consisting of R⁴ and halogen;

R² is halogen; straight- or branched-chain alkyl having up to six carbon atoms which is substituted by one or more —OR⁵; cycloalkyl having from three to six carbon atoms; or a member selected from the group consisting of nitro, cyano, —CO₂R⁵, —NR⁵R⁶, —S(O)ₚ R⁷, —O(CH₂)ₘOR⁵, —COR⁵, —N(R⁸)SO₂R⁷, —OR⁷, —OH, —OSO₂R⁷, —(CR⁹R¹⁰)ₜSO_{q}R^{7a}, —CONR⁵R⁶, —N(R⁸)—C(Z)Y, —(CR⁹R¹⁰)NR⁸R¹¹ and R⁴;

n is zero or an integer from one to three; when n is greater than one, then the groups R² are the same or different;

m is one, two or three;

p is zero, one or two;

q is zero, one or two;

t is an integer from one to four;

R³ is straight- or branched-chain alkyl group containing up to six carbon atoms which is un-substituted or which has one or more substituents selected from the group consisting of halogen, —OR⁵, —CO₂R⁵, —S(O)ₚR⁷, phenyl or cyano; or phenyl which is unsubstituted or which has one or more substituents selected from the group consisting of halogen, —OR⁵ and R⁴;

R⁴ is straight- or branched-chain alkyl, alkenyl or alkynyl having up to six carbon atoms which is un-substituted or is substituted by one or more halogen;

R⁵ and R⁶, which are the same or different, are each hydrogen or R⁴;

R⁷ and R^{7a} independently are R⁴, cycloalkyl having from three to six ring carbon atoms, or —(CH₂)_{w}-phenyl wherein phenyl is un-substituted or is substituted by from one to five R¹² which are the same or different;

w is zero or one;

R⁸ is hydrogen; straight- or branched-chain alkyl, alkenyl or alkynyl having up to ten carbon atoms which is un-substituted or is substituted by one or more halogen; cycloalkyl having from three to six ring carbon atoms; —(CH₂)_{w}-phenyl wherein phenyl is un-substituted or is substituted by from one to five R¹² which are the same or different; or —OR¹³;

R⁹ and R¹⁰ independently are hydrogen or straight- or branched-chain alkyl having up to six carbon atoms which is un-substituted or is substituted by one or more halogen;

R¹¹ is —S(O)_{q}R⁷ or —C(Z)Y;

R¹² is halogen; straight- or branched-chain alkyl having up to three carbon atoms which is un-substituted or is substituted by one or more halogen; or a member selected from the group consisting of nitro, cyano, —S(O)ₚR³ and —OR⁵;

Y is oxygen or sulphur;

Z is R⁴, —NR⁸R¹³, —NR⁸—NR¹³R¹⁴, —SR⁷ or —OR⁷; and

R¹³ and R¹⁴ independently are R⁸,

or an agriculturally acceptable salt or metal complex thereof,

The process for preparing a compound of the above formula (I) comprises:

(i) reacting a compound of formula (II) 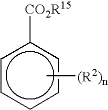
wherein R¹⁵ is a straight- or branched-chain alkyl group having up to six carbon atoms with a compound of formula (III) 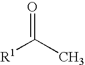
in an aprotic solvent in the absence of a base to form a compound of formula (IV) 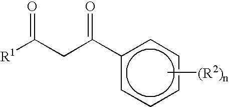
(ii) reacting a compound of formula (IV) with a compound that contains a leaving group L [such as alkoxy or N,N-dialkylamino, esp. ethoxy and CH(OCH₂ CH₃)₃ to form a compound of formula (V) 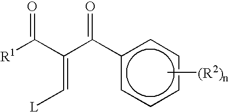
(iii) reacting a compound of formula (V) with hydroxylamine or a salt of hydroxylamine to form a compound of formula (I),

wherein the process further comprises producing the compound of formula (III) by:

providing gas phase raw materials, in a substantial theoretical stoichiometric ratio, to the converging section of a converging diverging funnel mixer;

wherein said gas phase raw materials are mixed by the significantly increased velocity and turbulent flow as they pass through the converging section and approach the transition section of the convergent divergent funnel mixer;

wherein said mixed raw materials pass through and out of the diverging section of the funnel mixer and into the tube reactor;

wherein the available reaction volume (ARV) of the tube reactor contains a super-layer catalyst;

wherein the mixed raw materials, in a substantial theoretical stoichiometric ratio pass through the catalyst, in the available reaction volume (ARV), to product the desired organic compound;

separating and recovering the desired organic compound.

In the above process, the compound of formula (III) is a ketone produced in accordance with an enhanced method and improved apparatus, device or devices, for preparing various organic compounds, such as ketones in accordance with of the present invention.

### EXAMPLE

The enhanced method and improved apparatus, device or devices, for preparing various organic compounds can also be used, in combination with other process steps, to prepare a compound of the following formula (X)

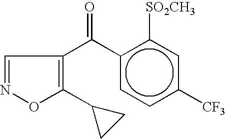

The specific process steps comprise:

(i) reacting a compound of formula (XI) 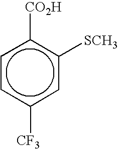
with a compound of formula (XII) 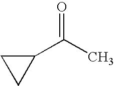
to form a compound of formula (XIII) 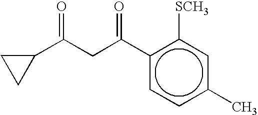
(ii) reacting a compound of formula (XIII) with CH(OCH₂CH₃)₃ to form a compound of formula (XIV) 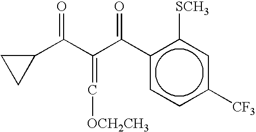
(iii) reacting a compound of formula (XIV) with hydroxylamine or a salt of hydroxylamine to form a compound of the formula (XV) 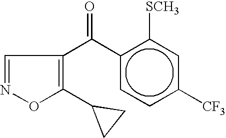
(iv) reacting a compound of formula (XV) with chloroperbenzoic acid [or an equivalent] to form a compound of the formula (X);

wherein the process further comprises producing the compound of formula (XII) by;

using an enhanced method and improved apparatus, device or devices, for preparing various organic compounds, such as ketones in accordance with of the present invention.

In the above process, the compound of formula (XII) is methyl cyclopropyl ketone (MCPK).

Further details of compounds of formula (I) and formula (X) described above are known in the art and described in one or more of PCT Publication No. WO 99/02476, U.S. Pat. No. 5,366,957 and U.S. Pat. No. 5,849,928; the substance of which is incorporated herein, by reference.

Although, the description of the preferred embodiment and method has been quite specific, it is contemplated that various modifications to the apparatus, device or devices, could be made without deviating from the spirit of the present invention. Nozzles, Injection Mixers, Vortex Mixers, Fan Blades and Baffles are examples of other types of mixing devices. Accordingly, it is intended that the scope of the present invention be dictated by the appended claims, as well as the description of the preferred embodiment.

1. An enhanced method and improved apparatus, device or devices, for preparing various organic compounds, such as: acids, aldehydes, amides, esters, ethers, and ketones comprising the steps of:
providing gas phase raw materials, in a substantial theoretical stoichiometric ratio, to a mixing device or devices, attached to a tube reactor;
wherein said gas phase raw materials are mixed;
wherein said mixed raw materials pass through and out of the apparatus, device or devices, and into the tube reactor;
wherein the available reaction volume (ARV) of the tube reactor contains a super-layer catalyst;
wherein the mixed raw materials, in a substantial theoretical stoichiometric ratio pass through the catalyst, in the available reaction volume (ARV), to product the desired organic compound;
separating and recovering the desired organic compound.

2. The method of claim 1;
wherein the apparatus, device or devices, is a converging diverging funnel;
wherein the gas phase raw materials, in a substantial theoretical stoichiometric ratio, are mixed by the significantly increased velocity and turbulent flow as they pass through the converging section and approach the transition section of the converging funnel;
wherein said mixed raw materials pass into, through and out of the diverging section of the funnel and into the tube reactor.
wherein the available reaction volume (ARV) of the tube reactor contains a super-layer catalyst;
wherein the mixed raw materials, in a substantial theoretical stoichiometric ratio pass through the catalyst, in the available reaction volume (ARV), to product the desired organic compound;
separating and recovering the desired organic compound.

3. The method of claim 2; by providing a gas phase raw material feed comprised of a first carboxylic acid or aldehyde or their derivatives and a second carboxylic acid to product the desired organic compound.

4. A method of claim 3; wherein the desired organic compound is a ketone.

5. A method of claims 3 and 4; wherein the first and second carboxylic acids are the same, iso-butyric acid and the symmetrical ketone is di-isopropyl ketone (DIPK)

6. A method of claim 3 and 4; wherein the first raw material is a carboxylic acid, such as acetic acid; the second carboxylic acid is nepetalic acid and the unsymmetrical ketone is; 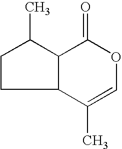
5,6,7,7a-tetrahydro-4,7-dimethylcyclopenta[c]pyran-1(4aH)-one;
or has the formula; 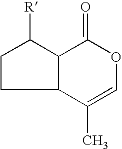
where R′ is a hydrocarbon substituents or other straight or branched-chain alkyl group having up to six carbon atoms.

7. The method of claim 2 by providing a gas phase raw material feed comprised of an amine and a carboxylic acid or aldehyde or their derivatives to product the desired organic compound.

8. A method of claim 7 wherein the desired organic compound is an amide.

9. A method of claims 7 and 8; wherein the raw materials are diethylamine and meta-toluic acid and the amide is N,N′-di-(ethyl)-meta-toluamide (DEET).

10. The method of claim 2 by providing a gas phase raw material feed comprised of an oxygen source and a compound of formula R¹—CX, where X is a group that leaves upon reaction; to product the desired organic compound in the form R¹—COH;
wherein R¹ is phenyl, which is un-substituted or substituted by one or more identical or different radicals selected from (C₁-C₁₂)-alkyl, (C₁-C₁₂)-alkoxy, (C₁-C₁₂)-alkanoyloxy, (C₁-C₁₂)-alkanoyl, amino, hydroxyl, —CH₂—O—(C₁-C₁₂)-alkyl, —NH—(C₁-C₁₂)-alkyl, —NH—CO—(C₁-C₁₂)-alkyl, or —S—(C₁-C₁₂)-alkyl;

11. A method of claim 10; wherein the desired organic compound is an aldehyde.

12. A method of claim 2 by providing a gas phase raw material feed comprised of a carboxylic acid or aldehyde or their derivatives and an alcohol to product the desired organic compound.

13. A method of claim 12; wherein the desired organic compound is an ester.

14. A method of claims 12 and 13; wherein the raw materials are benzoic acid and benzyl alcohol; and the ester is benzyl benzoate.

15. A method of claims 4, 6, 7, 8, 9, 11, and 13 for using the organic compounds, such as ketones, amides, aldehydes, and esters, in the preparation of insect repellents, animal repellents, herbicidal or other agricultural compounds and as flavor and/or fragrances ingredients.

16. A method of claims 1 and 2 for continuous preparation of an organic compound by providing a plurality of mixing devices, such as or converging diverging funnel, each attached to separate tube reactors.

17. A method of claim 4, wherein the desired organic compound is a ketone, used in a process for the preparation of a compound of formula (I) 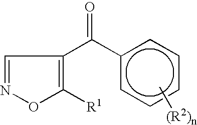
wherein:
R¹ is cycloalkyl having from three to six ring carbon atoms which is un-substituted or which has one or more substituents selected from the group consisting of R⁴ and halogen;
R² is halogen; straight- or branched-chain alkyl having up to six carbon atoms which is substituted by one or more —OR⁵; cycloalkyl having from three to six carbon atoms; or a member selected from the group consisting of nitro, cyano, —CO₂R⁵, —NR⁵R⁶, —S(O)ₚR⁷, —O(CH₂)ₘOR⁵, —COR⁵, —N(R⁸)SO₂R⁷, —OR⁷, —OH, —OSO₂R⁷, —(CR⁹R¹⁰)ₜSO_{q}R^{7a}, —CONR⁵R⁶, —N(R⁸)—C(Z)Y, —(CR⁹R¹⁰)NR⁸R¹¹ and R⁴;
n is zero or an integer from one to three; when n is greater than one, then the groups R² are the same or different;
m is one, two or three;
p is zero, one or two;
q is zero, one or two;
t is an integer from one to four;
R³ is straight- or branched-chain alkyl group containing up to six carbon atoms which is un-substituted or which has one or more substituents selected from the group consisting of halogen, —OR⁵, —CO₂R⁵, —S(O)ₚR⁷, phenyl or cyano; or phenyl which is unsubstituted or which has one or more substituents selected from the group consisting of halogen, —OR⁵ and R⁴;
R⁴ is straight- or branched-chain alkyl, alkenyl or alkynyl having up to six carbon atoms which is un-substituted or is substituted by one or more halogen;
R⁵ and R⁶, which are the same or different, are each hydrogen or R⁴;
R⁷ and R^{7a} independently are R⁴, cycloalkyl having from three to six ring carbon atoms, or —(CH₂)_{w}-phenyl wherein phenyl is un-substituted or is substituted by from one to five R¹² which are the same or different;
w is zero or one;
R⁸ is hydrogen; straight- or branched-chain alkyl, alkenyl or alkynyl having up to ten carbon atoms which is un-substituted or is substituted by one or more halogen; cycloalkyl having from three to six ring carbon atoms; —(CH₂)_{w}-phenyl wherein phenyl is un-substituted or is substituted by from one to five R¹² which are the same or different; or —OR¹³;
R⁹ and R¹⁰ independently are hydrogen or straight- or branched-chain alkyl having up to six carbon atoms which is un-substituted or is substituted by one or more halogen;
R¹¹ is —S(O)_{q}R⁷ or —C(Z)Y;
R¹² is halogen; straight- or branched-chain alkyl having up to three carbon atoms which is un-substituted or is substituted by one or more halogen; or a member selected from the group consisting of nitro, cyano, —S(O)ₚR³ and —OR⁵;
Y is oxygen or sulphur;
Z is R⁴, —NR⁸R¹³, —NR⁸—NR¹³R¹⁴, —SR⁷ or —OR⁷; and
R¹³ and R¹⁴ independently are R⁸,
or an agriculturally acceptable salt or metal complex thereof,
which process comprises:
(i) reacting a compound of formula (II) 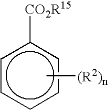
wherein R¹⁵ is a straight- or branched-chain alkyl group having up to six carbon atoms with a compound of formula (III) 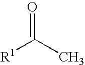
in an aprotic solvent in the absence of a base to form a compound of formula (IV) 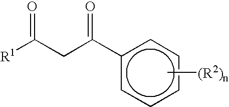
(ii) reacting a compound of formula (IV) with a compound that contains a leaving group L to form a compound of formula (V) 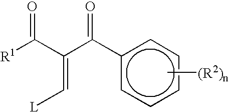
(iii) reacting a compound of formula (V) with hydroxylamine or a salt of hydroxylamine to form a compound of formula (I),
wherein the process further comprises using claims 3 and 4 to produce a compound of formula (III).

18. A method of claim 4, wherein the desired organic compound is a ketone, used in a process for the preparation of a compound of formula (X); 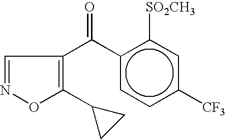
The specific process steps comprise:
(i) reacting a compound of formula (XI) 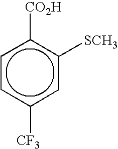
with a compound of formula (XII) 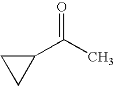
to form a compound of formula (XIII) 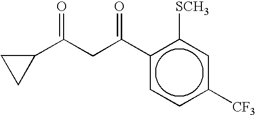
(ii) reacting a compound of formula (XIII) with CH(OCH₂ CH₃)₃to form a compound of formula (XIV) 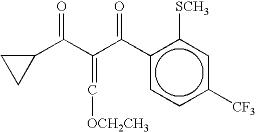
(iii) reacting a compound of formula (XIV) with hydroxylamine or a salt of hydroxylamine to form a compound of the formula (XV) 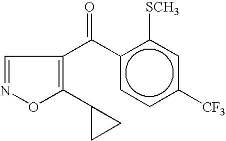
(iv) reacting a compound of formula (XV) with chloroperbenzoic acid [or an equivalent] to form a compound of the formula (X);
wherein the process further comprises producing the compound of formula (XII) by;
using claims 3, and 4; in accordance with of the present invention to produce methyl cyclopropyl ketone (MCPK), as the compound of formula (XII).
